(19) United States
(12) Plant Patent Application Publication
(10) Pub. No.: US 2012/0066804 P1
(43) Pub. Date: Mar. 15, 2012

(54) Clematis plant named 'Irene'

(50) ;

(76) Inventor: James P VanLaeken, Walworth, NY (US)

(21) Appl. No.: 12/807,753

(22) Filed: Sep. 14, 2010

(51) Int. Cl. A01H 5/00

(52) U.S. Cl. PLT/228

(57) ABSTRACT

A new variety of clematis plant named ‘Irene’. The variety is characterized by it's very large group of stamens almost ⅓ of the size of the flower. It has a beautiful contrast between the rose pink tepals and the white to cream flower center.

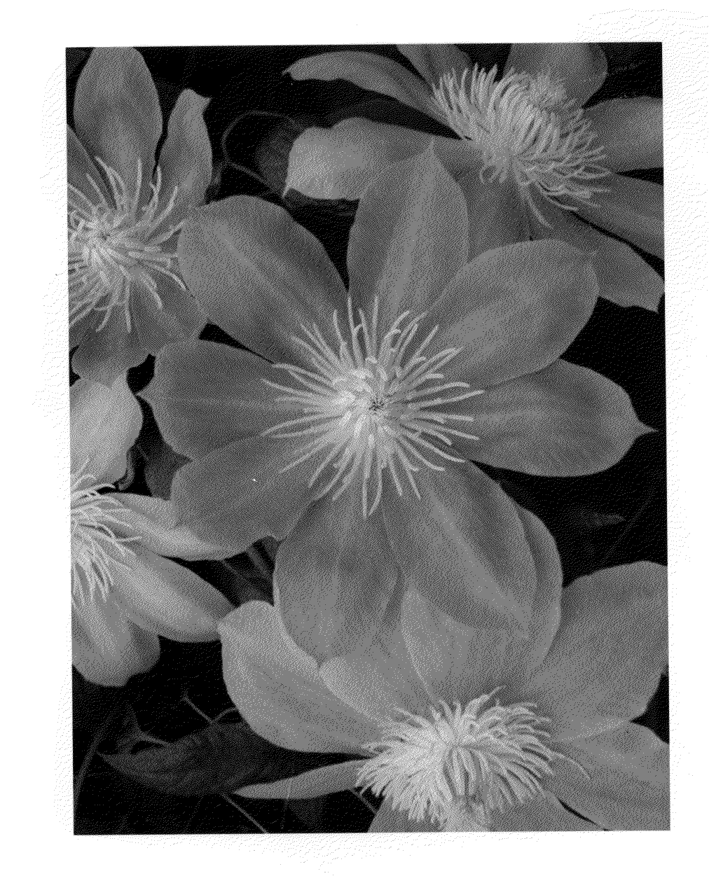

### BOTANICAL CLASSIFICATION

Clematis sp.

### CULTIVAR DESIGNATION

‘Irene’

### BACKGROUND OF THE VARIETY

The present invention relates to a new and distinct variety of hybrid clematis plant known by the cultivar name ‘Irene’.

The new variety was a cross that took place at a clematis nursery in Walworth, N.Y. in 2003 when Clematis sp. Corona was crossed with pollen from Clematis sp. Wada's Primrose. Seeds were planted and this variety was picked out because of it's unique color and extra large creamy white stamens. This variety was asexually propagated and observed over the next five years. Asexual propagation by vegetative cuttings has shown that the propagules are identical to the original plant and all distinguished characteristics. The stability of the variety is evidenced by the distinguished characteristics remaining true to form from generation to generation.

### SUMMARY OF THE INVENTION

Among the features which distinguish the new variety from other present available and known commercial clematis cultivars are the unique color of the flower. The flower has rose pink tepals with large stamens. It's compact habit and profuse blooming makes this variety a very desirable plant.

### RELEVANT PRIOR ART

The new variety has retained the compact growth habit and the prolific blooming of the mother Clematis sp. Corona as illustrated in Spring Valley Greenhouse, Inc. (6th Edition) on page 10 and the white cream stamens of the father Clematis sp. Wada's Primrose as illustrated in Spring Valley Greenhouse, Inc. (6th Edition) on page 6.

### BRIEF DESCRIPTION OF THE PHOTOGRAPH

The accompanying photograph illustrates the overall appearance of the new variety. The flowering and foliage colors as shown are, within the limitations of color photography, accurate and true reproductions of the actual colors of new variety. The photographic view is a front elevation of typical Clematis sp. ‘Irene’.

### BOTANICAL DESCRIPTION

The following information is based on a three year old plant grown in 50% shade in a five gallon container in a covered greenhouse with 60° F. ambient night temperatures and 75° F. ambient day temperatures in Walworth, N.Y. Color references are based on using the PANTONE® matching system.
1. Plant:
   A. Form.—upright climbing or spreading.
   B. Growth.—vigorous, vining 1. Height: approximately 4 feet to 6 feet 2. Diameter/Spread: approximately 2 feet to 3 feet.
   C. Foliage.—deciduous, compound-trifoliate, opposite 1. Size, typical and observed: a. compound leaf length 4 inches, width 3 inches b. individual leaflets length 2 to 2.5 inches, width 1 to 1.5 inches c. leaflets per compound leaf: 3 2. Quantity of leaves per stem: typically and observed: about 24 3. Color a. upper surface: dark green (PANTONE® #575U) b. lower surface: medium green (PANTONE® #576U) 4. Shape a. leaflets: base is rounded, margin is untoothed with pubescent edge, apex is pointed 5. Texture a. upperside: smooth and veined b. underside: ribbed and pubescent on veins and edges c. veination pattern: leaflets palmatic with green coloration on upper and lower 6. Petiole a. color: light green (PANTONE® #582U) b. length, typical and observed: 1 to 2 inches c. diameter, typical and observed: 1/16 inch d. glabrous: no.
   D. Wood.—1. New Shoots a. color: medium green (PANTONE® #575C) b. bark: pubescent c. internodes: cylindrical, approximately 2 to 5 inches in length and ⅛ inch in diameter 2. Old Wood a. color: medium to dark brown (PANTONE® #4695C) b. bark: smooth ⅛ inch in diameter c. internodes: cylindrical, approximately 5 to 6 inches in length and ⅛ inch in diameter and pubescent.
   E. Hardiness.—US Zones 4 through 8 1. low temperature tolerance, typical and observed: −10° F. to 0° F. 2. high temperature tolerance, typical and observed: 90° F. to 110° F.; requires 6 to 8 weeks dormancy at 35° F. to 40° F. to remain vigorous.
   F. Disease.—1. subject to any disease that affects clematis b. wilt c. botrytis d. powdery mildew.
   G. Pests.—1. subject to any pest that normally affect clematis a. aphids b. spider mites e. slugs and snails.
II. Flower:
   A. Blooming habit.—May and June then August & September based on climate in Rochester, N.Y. area.
   B. Bud.—1. Size: typical and observed from about 1.5 to 2 inches in length and about ½ to 1 inch in diameter 2. Form: ovate base with pointed tip 3. Color: light green with white veins (PANTONE® #583U).
   C. Stem.—1. Dimension, typical and observed: length 3 to 4 inches, diameter 1/16 inch 2. Aspect: vertical 3. strength: erect 4. Color: green (PANTONE® #378C).
   D. Bloom.—1. Size: typical and observed from about 5 to 6 inches in diameter with a depth of about ¾ inch 2. Form: round shaped with 6 to 8 tepals of equal size slightly overlapping 3. Color upper surface: medium rose pink (PANTONE® #218U) fading to a lighter rose pink (PANTONE® #233U) 4. Color of lower surface: outer edge is light pink (PANTONE® #2365U) middle is white with prominent veins 5. Characteristics: born on 1.5 to 2 inch stems on new and old growth 6. Quantity: 1 bloom per stem from 2 or 3 to several hundred blooms per plant depending on the age and size of plant 7. lastingness: 3 to 6 days as a cut flower or 7 to 10 days on plant.
   E. Tepals.—1. Form: elliptical shape, narrow at base rounding at mid section width 1.25 inches to 1.5 inches at widest point and then tapering down to a pointed apex. The overall tepal length being 2.5 to 3 inches. 2. Arrangement of tepals: regularly arranged slightly overlapping 3. Fragrance: none 4. Color: (see II D 3 and II D 4 above).
III. Reproductive organs:
   A. Stamens, filaments and anthers.—1. Arrangement: regularly arranged 2. Stamens: typical and observed 60 to 75 per bloom, length 0.75 inch 3. Anthers: length ¼ to ⅜ inch (PANTONE® #607U) yellowish 4. Filament: white.
   B. Pollen.—1. Color: white 2. Amount: abundant.
   C. Styles.—white, even, average length.
   D. Stigmas: 1. Color: white.
   E. Seed, typical and observed.—1. Diameter: 1/16 inch 2. Thickness: 1.32 inch 3. Color: (PANTONE® #577U) medium green 4. Shape: round 5. Quantity per bloom: 30 to 40 6. Description: ¾ inch long persistent style which is clothed with silky white hairs.

I claim:

1. A new and distinct variety of clematis plant named ‘Irene’ as shown and described herein.
